(19) United States
(12) Patent Application Publication
(10) Pub. No.: US 2010/0286511 A1
(43) Pub. Date: Nov. 11, 2010

(54) METHOD FOR DISPLAYING IMAGE DATA OF A PART OF A PATIENT'S BODY

(76) Inventors: Swen WOERLEIN, Munich (DE); Anke Weissenborn, Numberg (DE); Ingmar Thiemann, Munich (DE); Martin Mathis, Aying-Grosshelfendorf (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/774,876

(22) Filed: May 6, 2010

Related U.S. Application Data
(60) Provisional application No. 61/176,125, filed on May 7, 2009.

(30) Foreign Application Priority Data
May 6, 2009 (EP) 09 159 533.0

(51) Int. Cl. A61B 6/00

(52) U.S. Cl. 600/431

(57) ABSTRACT

The invention relates to a method for displaying image data of a part of a patient's body, in which the part of the patient's body comprising regions which are provided with a radiation-emitting contrast agent is detected in a first step by means of a detection device, wherein during the first step, the intensity of the radiation emitted by the regions provided with the contrast agent is higher than the intensity of other radiation which can be detected by the detection device and which is emitted by the detected part of the patient's body, wherein a patient image data set is produced on the basis of the detected radiation, and wherein the patient image data set is displayed to a user by means of an output device in a second step. The invention also relates to a device for displaying image data of a part of a patient's body, comprising: a detection device which comprises at least one camera for detecting radiation emitted by regions of the part of the patient's body which are provided with a radiation-emitting contrast agent; a computational unit which produces a patient image data set on the basis of the images detected by the detection device; and an output device for displaying the patient image data set produced.

### RELATED APPLICATION DATA

This application claims the priority of U.S. Provisional Application No. 61/176,125, filed on May 7, 2009, which is hereby incorporated in its entirety by reference.

### FIELD OF THE INVENTION

The invention relates to a method for displaying image data of a part of a patient's body and to a device for this purpose. In particular, the invention relates to a method in which in a first step, an image is recorded of a part of a patient's body which has been provided with a fluorescent contrast agent, and in a subsequent second step, recorded image data is displayed to a user. The invention also relates to a corresponding device for performing this method.

### BACKGROUND OF THE INVENTION

Methods in which particular regions of interest within a part of a patient's body, for example tumor tissue, are marked by means of a fluorescent contrast agent are known from the prior art. Since the contrast agent is adsorbed particularly well onto the tumor tissue, the tumor tissue luminates more brightly than the surrounding healthy tissue, under particular lighting conditions. If the tumor tissue is to be surgically removed, then only the brightly luminating regions of the part of the patient's body have to be removed. However, in order to clearly visualize the fluorescent radiation emitted by the fluorescent substances, it is often necessary to generate particular lighting conditions in the operating room. If the identified tumor tissue is subsequently to be removed, then the operating theatre lighting necessary for operations has to be switched on for this purpose. By repeating these steps, it is possible to also remove any tumor tissue still remaining on the patient. It is thus necessary to constantly switch back and forth between the operating theatre lighting and the particular lighting conditions necessary for visualizing the tumor tissue which is to be removed. This significantly disrupts the normal flow of an operation.

It is also often a problem if the luminosity of the contrast agent rapidly decreases within a short period of time or if the contrast agent is constantly being borne away by the bloodstream. In these cases, new contrast agent has to be constantly supplied to the part of the patient's body.

### SUMMARY OF THE INVENTION

It is an object of the invention to provide a method and a device, with the aid of which regions of a part of a patient's body can be marked by means of a contrast agent, such that they are clearly visible for a sustained period of time, without disrupting the flow of an operation under normal lighting conditions.

This object is solved by a method for displaying image data of a part of a patient's body, in which the part of the patient's body comprising regions which are provided with a radiation-emitting contrast agent is detected in a first step by means of a detection device, wherein during the first step, the intensity of the radiation emitted by the regions provided with the contrast agent is higher than the intensity of other radiation which can be detected by the detection device and which is emitted by the detected part of the patient's body, wherein a patient image data set is produced on the basis of the detected radiation, and wherein the patient image data set is displayed to a user by means of an output device in a second step, and is solved by a device for displaying image data of a part of a patient's body, comprising: a detection device which comprises at least one camera for detecting radiation emitted by regions of the part of the patient's body which are provided with a radiation-emitting contrast agent; a computational unit which produces a patient image data set on the basis of the images detected by the detection device; and an output device for displaying the patient image data set produced.

In the method in accordance with the invention, a part of a patient's body comprising regions which are provided with a radiation-emitting contrast agent is detected in a first step by means of a detection device, wherein during the first step, the intensity of the radiation emitted by the regions provided with the contrast agent is higher than the intensity of other radiation which can be detected by the detection device and which is emitted by the detected part of the patient's body, wherein a patient image data set is produced on the basis of the detected radiation, and wherein the patient image data set is displayed to a user by means of an output device in a second, in particular subsequent step.

In other words, a part of a patient's body is thus supplied with a contrast agent which emits radiation and thus visualizes regions in which the contrast agent is particularly well adsorbed. If the contrast agent emits radiation in the visible light spectrum, these regions will appear brighter to the naked eye than other regions in which less contrast agent or no contrast agent is adsorbed. Such a contrast agent can for example be ALA (aminolevulinic acid) for marking tumor tissue or ICG (indocyanine green) for marking blood vessels. A contrast agent which emits radiation in the infrared range or the ultraviolet light range is also conceivable.

In order to provide contrast agent to the regions to be indicated, a single or repeated injection of contrast agent is conceivable, wherein the contrast agent is independently adsorbed onto particular regions of interest or preferably predominates in or at particular regions of interest. A continuous infusion of contrast agent is also conceivable. Manually applying contrast agent to particular regions which are to be indicated or manually introducing contrast agent into particular regions which are to be indicated would also be conceivable.

The part of the patient's body comprising the regions which are provided with contrast agent is subsequently detected and/or recorded by means of a detection device, advantageously at a time shortly after the regions to be indicated have been provided with the contrast agent, since the emission strength of the contrast agent is very high at this time, and high-contrast recordings of the part of the patient's body comprising the indicated regions are therefore possible. If the emission strength of the contrast agent deteriorates over time or if the contrast agent is borne away by flowing body fluids, then the need for detection to be performed by the detection device as shortly as possible after the regions to be indicated have been provided with contrast agent becomes clear. In summary, it may thus be said that the part of the patient's body is detected at a time at which the radiation intensity of the contrast agent is higher than the intensity of other radiation emitted by the part of the patient's body. High-contrast recordings of the part of the patient's body, with regions marked such that they are clearly visible, are the result.

A patient image data set is produced on the basis of the images of the part of the patient's body detected by the detection device, wherein one or more recorded images can be aligned with regard to their location and alignment relative to the actual part of the patient's body. Individual recorded images can also be aligned relative to each other.

The advantage of the method in accordance with the invention is seen in particular in the second step which then follows, i.e. when the patient image data set is displayed by means of an output device. Despite the fact that the emission strength and/or luminosity of the contrast agent could have significantly decreased since the indicated regions were provided with contrast agent, which makes identifying these regions more difficult, the method in accordance with the invention provides the user with high-contrast images, comprising clearly delineated regions marked by contrast agents, at any subsequent point in time. It is also possible to display the produced images by means of an output device, in normal lighting conditions in the operating theatre during an operation, without being bound by specific lighting conditions such as for example a dimmed operating theatre. High-contrast images of the part of the patient's body, comprising the regions marked by contrast agent, are thus provided even while an operating theatre is brightly lit.

In a preferred embodiment, the contrast agent emits radiation in the visible light spectrum. It is however also in principle conceivable for the contrast agent to emit non-visible radiation such as for example x-ray radiation, radioactive radiation (for example, a radiation, β radiation and/or γ radiation), infrared radiation or ultraviolet radiation. It is in principle also conceivable for the contrast agent to comprise a magnetic material which can be detected on the basis of the magnetic properties of the material.

It is also conceivable for individual regions of the part of the patient's body which are to be marked to be provided with a number of different contrast agents which emit respectively different or similar radiation or exhibit different properties. It is thus possible to produce recordings of the part of the patient's body by means of different imaging methods, wherein in said recordings, the regions to be marked are clearly delineated from the remaining regions of the part of the patient's body.

In another preferred embodiment, the patient image data set comprises the surface data of the part of the patient's body. It is in principle possible to provide the regions of the part of the patient's body which are to be marked with a radiation-emitting contrast agent. If a direct visual contact between the detection device and the contrast agent and/or the regions provided with the contrast agent is necessary in order for the emitted radiation to be detected by the detection device, then detection only makes sense if the radiation to be detected is emitted from surface parts on the part of the patient's body. As soon as the emitted radiation is also to be detectable through patient tissue, however, it is also possible to detect regions which are situated within the part of the patient's body and provided with contrast agent, without direct visual contact, by means of the detection device. This would for example be the case when the contrast agent emits x-ray radiation or radioactive radiation. A contrast agent comprising magnetic material could also in principle be detected within the part of the patient's body by a detection device arranged outside the part of the patient's body, without any direct visual contact.

The patient image data set can also comprise a multitude of images of the part of the patient's body which are detected at different times. It is thus conceivable for a series of images of the part of the patient's body to be produced in a chronological sequence from the same viewing angle, such that a chronological progression can be identified in the patient image data set. This chronological progression can for example identify movements of joints or a change in the location or radiation intensity of the contrast agent. A video film recording of the part of the patient's body can in particular be produced which can for example be played back at a subsequent point in time in a continuous loop.

In another preferred embodiment, images or films of the part of the patient's body are produced simultaneously from at least two different viewing directions. If the position and alignment of the detection device is known and the spatial location of the viewing directions is also known, it is also possible to determine, by triangulation, the position and alignment of objects which can be identified on a number of images and assigned to each other. It is ultimately possible to produce, on the basis of these images, a 3D image data set from which it is possible to deduce the location and alignment of individual objects and also the location and alignment of the marked regions. Instruments which can be spatially located by a medical navigation system can thus be guided to the marked regions.

In another preferred embodiment, the patient image data set is superimposed onto a surgeon's field of view in the second step, by means of the output device. It is thus possible for the surgeon to be able to view the image data generated, without having to look away from the patient and/or the part of the patient's body, wherein the patient image data can for example be superimposed in the manner of a head-up display. Thus, as soon as the surgeon looks through such an apparatus comprising a head-up display, such as for example a pair of glasses or a microscope, he sees both the patient to be treated and the superimposed patient image data.

If the patient image data set was produced using a tracked detection device, then both the spatial position and the spatial alignment of the regions marked by the contrast agent is known. If the patient and/or the part of the patient's body has not been moved since the detection device detected the radiation emitted by the contrast agent and the patient image data set was produced, and if the position and alignment of the output device is also known, then the patient image data set can be congruently superimposed onto the actual part of the patient's body in the user's field of view. In this case, “congruently” is intended to mean that both the location and alignment and the image size of the image information stored in the patient image data set matches those of the actual patient and/or part of the patient's body in the surgeon's field of view. Thus, the surgeon for example sees the tumor tissue—marked by the contrast agent and indicated by brightly luminating regions—at exactly the point on the actual patient and/or part of the patient's body at which it is also situated on or in the actual patient and/or part of the patient's body. If the tumor tissue is to be removed or treated, the surgeon thus merely has to guide the corresponding medical instrument to the region on or in the part of the patient's body which is superimposed by the head-up display.

In accordance with another embodiment of the present invention, it is also possible to display the patient image data set together with at least one other patient image data set. It is thus conceivable for the patient image data set which is produced by the method in accordance with the invention and comprises the marked regions to be superimposed onto an already existing patient image data set such as for example an ultrasound, MRT, MRA or CT image data set or to be displayed in parallel with it. It is also conceivable within this context to correlate three-dimensional data sets or perform an MPR method. It is thus conceivable for a blood vessel which is marked by ICG to be placed over an MRA image data set in order to see the position of the marked blood vessel in the MRA environment. This also allows an assessment of the so-called brain shift or, in very general terms, changes in the position and location of the tissue during a surgical operation.

In accordance with another preferred embodiment, the detection device and/or the output device are detected by means of a medical navigation system, such that their spatial position and alignment are known. If both the detection device and the output device are detected, it is possible to display the image data detected by the detection device in a spatially correct position and alignment with respect to the actual patient and/or part of the patient's body, as has already been described further above. If the patient and/or part of the patient's body is also registered, i.e. if its spatial position and alignment are for example tracked by means of a navigation reference and a medical navigation system, it is possible to correctly superimpose the patient image data set over the actual patient, even if the patient and/or part of the patient's body intra-operatively moves. In very general terms, the patient and/or part of the patient's body could be moved after the radiation emitted by the contrast agent has been detected, without endangering the patient image data set being correctly superimposed over the patient and/or part of the patient's body.

It is also conceivable for the part of the patient's body to be illuminated by means of an illuminating device. It is thus possible, in the region of the part of the patient's body of which a patient image data set is to be produced, to generate illumination conditions which enable particularly clear and high-contrast images. It is thus in particular conceivable for the contrast agent to be irradiated with ultraviolet light, infrared light or visible light by the illuminating device, in order to excite the contrast agent to emit radiation.

Many contrast agents can in principle be used in connection with the present invention, such as for example radiation-emitting contrast agents or also contrast agents which comprise a magnetic substance, wherein the radiation-emitting contrast agents in particular include contrast agents which emit x-ray radiation or light. Within the range of light-emitting contrast agents, the emission of visible light, infrared light or ultraviolet light is also conceivable. Preferably, contrast agents which emit fluorescent radiation or phosphorescent radiation are used in connection with the present invention.

A method is also conceivable, in which the intensity of the radiation emitted by the regions marked with the contrast agent is lower during the second step, i.e. while displaying the patient image data set, than the intensity during the first step, i.e. while detecting the radiation emitted by the contrast agent and producing the patient image data set on the basis of the detected radiation. The fact that the patient image data set produced on the basis of the detected radiation can be stored for any length of time and can be displayed again at any desired point in time allows very clear and high-contrast images to be displayed even if the radiation intensity of the contrast agent has already long since decreased or the contrast agent no longer adheres to the part of the patient's body at the regions to be marked.

Even if the intensity of the radiation emitted by the marked regions is lower during the second step than the intensity of other optical radiation emitted by the detected part of the patient's body, very clear and high-contrast images can be displayed, as described above, in which the regions marked by the contrast agent are shown. Even if the operating theatre lighting is already switched on again during the second step and the radiation emitted by the contrast agent is therefore barely identifiable, the surgeon can clearly identify the regions marked by the contrast agent by displaying the patient image data set and does not, as before, have to switch off the operating theatre lighting in order to see the radiation emitted by the contrast agent.

The present invention also includes a device for displaying image data of a part of a patient's body, comprising: a detection device which comprises at least one camera for detecting radiation emitted by regions of the part of the patient's body which are provided with a radiation-emitting contrast agent; a computational unit which produces a patient image data set on the basis of the images detected by the detection device; and an output device for displaying the patient image data set produced.

In a preferred embodiment, the detection device detects radiation in the visible light spectrum. It is conceivable for the detection device to detect images of the part of the patient's body while the operating theatre lighting is switched off, wherein the radiation emitted by the contrast agent distinguishes the regions to be marked from the remaining tissue in a particularly clear and high-contrast way. Video cameras, infrared cameras and x-ray detectors are also conceivable in connection with the detection device.

In a preferred embodiment, the device in accordance with the invention comprises a detection device comprising at least two cameras which detect the part of the patient's body and in particular its surface from different viewing directions. In this way, features which can be identified on a number of images recorded from different viewing directions can be assigned to each other. If the spatial position and alignment of the cameras is known, it is also possible to deduce the spatial position of these features by means of triangulation. A detection device comprising at least two cameras thus enables a 3D patient image data set to be produced.

It is also possible for the detection device to detect a multitude of images at different times, wherein a video film of the part of the patient's body is in particular produced. Thus, moving image data of the part of the patient's body can be displayed to the surgeon at a subsequent point in time by means of an output device, and in particular superimposed into the surgeon's field of view.

It is also conceivable for the detection device and/or the output device to be detected by a medical tracking system. In particular, the detection device and/or the output device can comprise a navigation reference which is detected by a medical tracking system. In this way, it is possible to determine the position and alignment of the detection device and/or the output device, which for example enables the image data detected by the detection device to be superimposed exactly over the actual patient and/or part of the patient's body.

The device in accordance with the invention can also comprise an illuminating device which illuminates the surface of the part of the patient's body and in particular excites the contrast agent to emit. The illumination conditions can thus be adapted to the respective requirement of the detection device, such that images of the part of the patient's body which are as focused and high-contrast as possible are produced. In particular, the contrast agent situated at the part of the patient's body to be detected can be excited to emit radiation by being illuminated by means of the illuminating device, such as for example by being irradiated with ultraviolet radiation or infrared radiation.

It is also conceivable for the computational unit of the device in accordance with the invention to receive the image data detected by the detection device, produce a patient image data set—in particular, a surface image data set—of the part of the patient's body on the basis of the detected image data, and provide the patient image data set to the output device for displaying—in particular, at a subsequent point in time. The computational unit can also store the patient image data set produced or at least buffer it until it is displayed. The image data which is detected by the detection device and processed by the computational unit to form a patient image data set can thus be displayed to the surgeon at any subsequent point in time. Even if the radiation intensity of the contrast agent has since decreased or the contrast agent no longer adheres to the regions to be marked, the surgeon is provided with a high-contrast image of the part of the patient's body comprising the marked regions. The same applies if the operating theatre lighting has since been switched on again, such that the radiation emitted by the contrast agent is no longer identifiable.

It is also possible in connection with another embodiment for the computational unit to superimpose the image data detected by the detection device onto at least one other existing image data set of the same part of the patient's body. In other words, the computational unit produces a composite image consisting of the image data detected by means of the detection device and an already existing patient image data set, for example an ultrasound image data set, an MRT image data set, an MRA image data set or a CT image data set.

The device in accordance with the invention can also comprise an optical observation device as the output device, which can in particular be a medical microscope which specifically projects the image data detected by the detection device and/or the patient image data set into a user's field of view. The medical microscope can comprise a head-up display which superimposes the images which are detected by the detection device and processed to form a patient image data set onto the image of the part of the patient's body provided by the optical observation device and/or medical microscope. The surgeon who looks through the optical observation device and/or medical microscope thus sees the regions of the part of the patient's body which are highlighted by the contrast agent, embedded in the current actual part of the patient's body which can be seen through the optical observation device and/or medical microscope. Advantageously, the surgeon no longer has to look away from the patient and/or part of the patient's body to be treated in order to view the regions marked by the contrast agent.

It is however also possible for an output device to be provided in the form of a screen or computer monitor.

It is also conceivable for image data sets produced by other imaging methods to be displayed, superimposed in parallel or over the image data detected by the detection device.

A projector which projects the patient image data set onto a projection area or onto the surface of the part of the patient's body is also conceivable as the output device. The regions to be highlighted by the contrast agent are thus projected directly onto the surface of the patient's body, such that the surgeon performing the treatment does not have to look up from the site of the operation in order to view the regions to be highlighted by the contrast agent—for example, tumor tissue.

The contrast agent can also be a liquid contrast agent which is arranged in the part of the patient's body or on the surface of the part of the patient's body, wherein it is possible to supply the liquid contrast agent to the relevant part of the patient's body by means of an injection or infusion, wherein the contrast agent is preferably adsorbed at regions to be highlighted—for example, tumor tissue. If the contrast agent emits radiation, these regions can be distinguished from the surrounding tissue on the basis of their increased radiation intensity. Manually applying the contrast agent to the surface of the part of the patient's body is also conceivable.

In connection with the device in accordance with the invention, markers can also be arranged on the part of the patient's body and can in particular be markers which emit or reflect infrared light and are detected by the detection device. This is in particular advantageous when a medical tracking system is used in connection with the device in accordance with the invention and the patient and/or part of the patient's body is to be positionally located and registered. In this case, it is then possible to move the patient after the radiation emitted by the contrast agent has been detected and a patient image data set has been produced on the basis of the detected radiation, wherein the patient image data set produced can be congruently superimposed onto the actual patient and/or part of the patient's body, if the location and alignment of the detection device and the output device can also be detected by a medical tracking system.

These markers can also serve to superimpose the produced patient image data set onto the part of the patient's body in a spatially correct way. Thus, for example, the distance from the output device to the part of the patient's body can be directly determined on the basis of the distance between the markers. In this way, it is also possible to automatically set the focus of the microscope.

### BRIEF DESCRIPTION OF THE DRAWINGS

An embodiment of the present invention is described in more detail on the basis of the enclosed figures. The present invention can include any of the features described here, individually and in any expedient combination.

FIG. 1 shows an embodiment of the device in accordance with the invention.

FIG. 2 shows an embodiment of the method in accordance with the invention.

### DETAILED DESCRIPTION

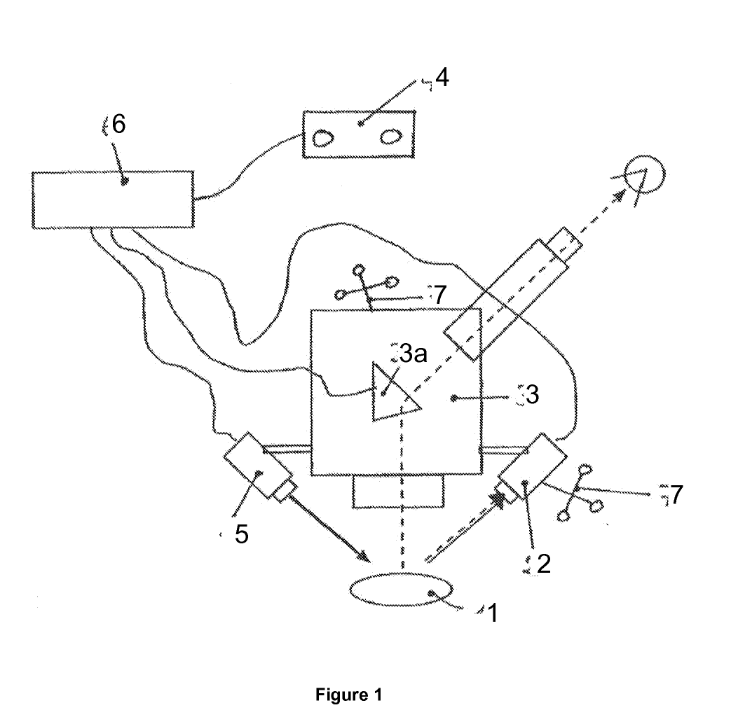

FIG. 1 shows a device in accordance with the invention for displaying image data of a part of a patient's body. The device in accordance with the invention initially comprises an output device 3 in the form of a medical microscope through which the surgeon looks in order to view a part of the patient's body 1. The optical radiation path which proceeds from the part of the patient's body 1 and leads to the surgeon's eye is shown in FIG. 1 by the broken-line arrow.

The medical microscope 3 also comprises a navigation reference 7, such that the spatial position and alignment of the medical microscope 3 can be determined by the tracking system 4, wherein the tracking system 4 comprises two schematically shown infrared cameras which detect the navigation reference 7, such that the position and alignment of the medical microscope 3 can be determined by means of the tracking system 4 and the navigation reference 7, by triangulation. An illuminating device 5 is fixedly connected to the medical microscope 3 and if required illuminates the part of the patient's body 1 to be detected and in particular excites the contrast agent situated at the part of the patient's body 1 to emit radiation. The illuminating radiation is shown in the figure by the continuous-line arrow.

A detection device 2 is also connected to the medical microscope 3 and detects the radiation emitted by the contrast agent on or in the part of the patient's body 1. This emitted and detected radiation is shown in FIG. 1 by the part broken-line, part continuous-line arrow. In the embodiment shown in FIG. 1, the detection device 2 also comprises a navigation reference 7, by means of which the spatial position and alignment of the detection device 2 can be determined. If the detection device 2 is fixedly connected to the microscope 3, which already comprises a navigation reference 7, then such a navigation reference 7 on the detection device 2 is not actually necessary. As soon as the detection device 2 can be spatially moved relative to the medical microscope 3, however, a navigation reference 7 of its own is necessary in order to determine the position and alignment of the detection device 2.

The detection device 2, the medical microscope 3, the navigation system 4 and the illuminating device 5 are respectively connected to a computational unit 6 via data lines. These data lines can be both cable-based and radio-based data lines.

In order to record an image of the part of the patient's body 1, the operating theatre lighting is for example switched off and, if required, the illuminating device 5 is instead switched on. The radiation emitted by the contrast agent on or in the part of the patient's body 1 is detected by the detection device 2 and relayed to the computational unit 6. A patient image data set is produced by the computational unit 6 on the basis of this image information and displayed via the head-up display 3a arranged in the centre of the medical microscope 3.

If a surgeon then looks through the medical microscope 3 in order to view the part of the patient's body 1, the produced patient image data set comprising the regions marked by the contrast agent can be superimposed into the surgeon's field of view by means of the head-up display 3a. Since the position and alignment of both the detection device 2 and the medical microscope 3 can be detected by the medical tracking system 4, using navigation references 7 in each case, it is also possible for the computational unit 6 to display the produced patient image data set, by means of the head-up display 3a, in such a way that the surgeon always sees the produced patient image data set congruently with respect to the actual part of the patient's body 1. In order for example to remove a tumor tissue which is marked by contrast agent, the surgeon merely has to remove the region of the part of the patient's body 1 which is superimposed with the region which is marked by the contrast agent and superimposed by means of the head-up display 3a.

It is also then possible to produce another patient image data set comprising the remaining tumor tissue, in order to assess the success of the operation. As applicable, the method in accordance with the invention can be repeated in order to remove the remaining tumor tissue.

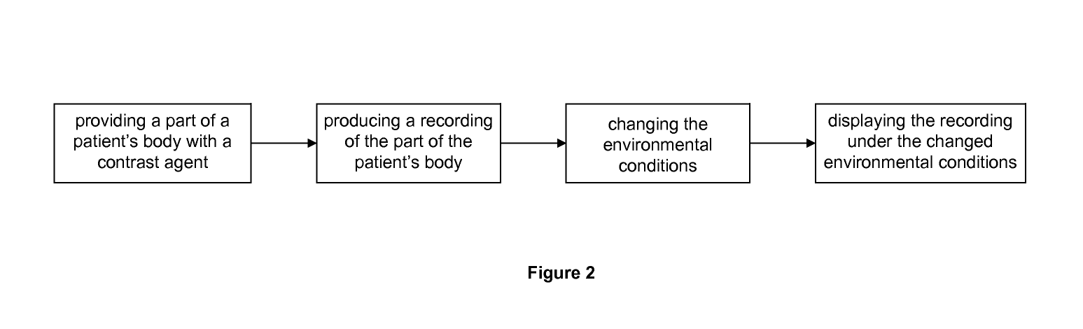

FIG. 2 shows an embodiment of the method in accordance with the invention in principle.

Firstly, a part of a patient's body is provided with a contrast agent, for example ALA for marking tumor tissue or ICG for marking blood vessels. A recording of this part of the patient's body is then produced by means of a detection device, wherein the regions marked by the contrast agent can be differentiated from the remaining tissue of the part of the patient's body on the basis of their higher radiation intensity. In order to produce such recordings, specific environmental conditions are in most cases necessary, for example operating theatre lighting which has been significantly reduced in brightness or even switched off completely. Only under such illumination conditions it is possible to identify the weak radiation emitted by the contrast agent and produce a clear and high-contrast image of the part of the patient's body which has been provided with the contrast agent. As soon as the images and/or a video film of the part of the patient's body have been produced under the specific environmental conditions, the environmental conditions can be adapted to the normal conditions in a following step. After the images have been produced, the intensity of the operating theatre lighting can again be set such that a normal flow of the operation is possible.

Since the radiation emitted by the contrast agent is no longer identifiable at this time, i.e. under normal operating theatre lighting, the recordings produced and/or the patient image data set produced are displayed by means of the output device in a following step.

Computer program elements of the invention may be embodied in hardware and/or software (including firmware, resident software, micro-code, etc.). The computer program elements of the invention may take the form of a computer program product which may be embodied by a computer-usable or computer-readable storage medium comprising computer-usable or computer-readable program instructions, “code” or a “computer program” embodied in said medium for use by or in connection with the instruction executing system. Within the context of this application, a computer-usable or computer-readable medium may be any medium which can contain, store, communicate, propagate or transport the program for use by or in connection with the instruction executing system, apparatus or device. The computer-usable or computer-readable medium may for example be, but is not limited to, an electronic, magnetic, optical, electromagnetic, infrared or semiconductor system, apparatus, device or medium of propagation, such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiment(s).

Although the invention has been shown and described with respect to one or more particular preferred embodiments, it is clear that equivalent amendments or modifications will occur to the person skilled in the art when reading and interpreting the text and enclosed drawing(s) of this specification. In particular with regard to the various functions performed by the elements (components, assemblies, devices, compositions, etc.) described above, the terms used to describe such elements (including any reference to a “means”) are intended, unless expressly indicated otherwise, to correspond to any element which performs the specified function of the element described, i.e. which is functionally equivalent to it, even if it is not structurally equivalent to the disclosed structure which performs the function in the example embodiment(s) illustrated here. Moreover, while a particular feature of the invention may have been described above with respect to only one or some of the embodiments illustrated, such a feature may also be combined with one or more other features of the other embodiments, in any way such as may be desirable or advantageous for any given application of the invention.

What is claimed is:

1. A method for displaying image data of a part of a patient's body, in which the part of the patient's body comprising regions which are provided with a radiation-emitting contrast agent is detected in a first step by means of a detection device, wherein during the first step, the intensity of the radiation emitted by the regions provided with the contrast agent is higher than the intensity of other radiation which can be detected by the detection device and which is emitted by the detected part of the patient's body, wherein a patient image data set is produced on the basis of the detected radiation, and wherein the patient image data set is displayed to a user by means of an output device in a second step.

2. The method according to claim 1, wherein the contrast agent emits radiation in the visible light spectrum.

3. The method according to claim 1, wherein the patient image data set comprises the surface data of the part of the patient's body.

4. The method according to claim 1, wherein the patient image data set comprises a multitude of images of the part of the patient's body which are detected at different times.

5. The method according to claim 4, wherein the multitude of images of the part of the patient's body are a video recording of the part of the patient's body.

6. The method according to claim 1, wherein in order to produce a 3D patient image data set, the part of the patient's body is detected from at least two different viewing directions by means of the detection device in the first step.

7. The method according to claim 1, wherein the patient image data set is superimposed onto a user's field of view by means of the output device in the second step.

8. The method according to claim 1, wherein the patient image data set is displayed together with at least one other patient image data set by means of the output device in the second step.

9. The method according to claim 8, wherein the patient image data set is superimposed onto at least one other patient image data set by means of the output device in the second step.

10. The method according to claim 1, wherein the detection device and/or the output device are detected by means of a medical navigation system.

11. The method according to claim 1, wherein the part of the patient's body is illuminated by means of an illuminating device.

12. The method according to claim 11, wherein the contrast agent is excited to emit radiation.

13. The method according to claim 1, wherein the radiation emitted by the contrast agent is fluorescent radiation or luminescent radiation.

14. The method according to claim 1, wherein the intensity of the radiation emitted by the regions provided with the contrast agent is lower during the second step than the intensity during the first step.

15. The method according to claim 1, wherein the intensity of the radiation emitted by the regions provided with the contrast agent is lower during the second step than the intensity of other optical radiation emitted by the detected part of the patient's body.

16. A device for displaying image data of a part of a patient's body, comprising:
a detection device which comprises at least one camera for detecting radiation emitted by regions of the part of the patient's body which are provided with a radiation-emitting contrast agent;
a computational unit which produces a patient image data set on the basis of the images detected by the detection device; and
an output device for displaying the patient image data set produced.

17. The device according to claim 16, wherein radiation is emitted in the visible light spectrum and detected by the detection device.

18. The device according to claim 16, wherein the detection device comprises at least two cameras which detect the part of the patient's body from different viewing directions.

19. The device according to claim 18, wherein the at least two cameras detect the surface of the part of the patient's body from different viewing directions.

20. The device according to claim 16, wherein the detection device detects a multitude of images at different times.

21. The device according to claim 20, wherein the multitude of images are a video of the part of the patient's body.

22. The device according to claim 16, wherein the detection device and/or the output device are detected by a medical tracking system.

23. The device according to claim 22, wherein the detection device and/or the output device comprise navigation references which are detected by a medical tracking system.

24. The device according to claim 16, also comprising an illuminating device which illuminates the surface of the part of the patient's body.

25. The device according to claim 24, wherein the illuminating device excites the contrast agent to emit.

26. The device according to claim 16, wherein the computational unit receives the image data detected by the detection device, produces a patient image data set of the part of the patient's body on the basis of the detected image data, and provides the patient image data set to the output device for displaying.

27. The device according to claim 26, wherein the patient image data set of the part of the patient's body is a surface image data set of the part of the patient's body.

28. The device according to claim 26, wherein the patient image data set is displayed at a subsequent point in time.

29. The device according to claim 16, wherein the computational unit superimposes the image data detected by the detection device onto at least one other existing image data set of the same part of the patient's body.

30. The device according to claim 16, wherein the output device is an optical observation device.

31. The device according to claim 30, wherein the optical observation device is a medical microscope.

32. The device according to claim 30, wherein the optical observation device projects the image data detected by the detection device into a user's field of view.

33. The device according to claim 16, wherein the output device is a screen.

34. The device according to claim 16, wherein the output device is a projector which projects the patient image data set onto the surface of the part of the patient's body.

35. The device according to claim 16, wherein the contrast agent is a liquid contrast agent which is arranged in the part of the patient's body or on the surface of the part of the patient's body.

36. The device according to claim 16, wherein the detection device detects markers arranged on the part of the patient's body.

37. The device according to claim 35, wherein the markers arranged on the part of the patient's body are markers which emit or reflect infrared light.

38. A computer program stored on a machine-readable medium for displaying image data of a part of a patient's body, comprising:
code which in a first step detects the part of the patient's body comprising regions which are provided with a radiation-emitting contrast agent by means of a detection device, wherein during the first step, the intensity of the radiation emitted by the regions provided with the contrast agent is higher than the intensity of other radiation which can be detected by the detection device and which is emitted by the detected part of the patient's body;
code which produces a patient image data set on the basis of the detected radiation; and
code which displays the patient image data set to a user by means of an output device in a second step.
